(19) United States
(12) Patent Application Publication
(10) Pub. No.: US 2015/0276758 A1
(43) Pub. Date: Oct. 1, 2015

(54) Biomarker Detection Device for Monitoring Peptide and Non-Peptide Markers

(71) Applicant: Anteneh Addisu, Ocala, FL (US)

(72) Inventor: Anteneh Addisu, Ocala, FL (US)

(21) Appl. No.: 14/483,987

(22) Filed: Sep. 11, 2014

Related U.S. Application Data
(60) Provisional application No. 61/973,658, filed on Apr. 1, 2014.

(51) Int. Cl. G01N 33/68

(52) U.S. Cl. CPC G01N 33/6854

(57) ABSTRACT

A biomarker detection device is a wearable device utilized to detect and analyze a sample of bodily fluid for the presence of various biomarkers that are indicative of a variety of medical conditions. A microneedle array is inserted into the user's skin in order to draw a sample of bodily fluid into an H-filter within the biomarker detection device. A reagent with antibodies is released into the H-filter as well and the antibodies are able to bond to biomarkers within the bodily fluid sample after the sample and the reagent have been mixed. A sensor connected to the H-filter comes into contact with the bonded antibodies and biomarkers. A computing unit electronically connected to the sensor detects the biomarkers bonded to the antibodies and performs analysis to determine if the level of biomarkers present in the acquired sample is unsafe or outside of a “normal” range.

The current application claims a priority to the U.S. Provisional Patent application Ser. No. 61/973,658 filed on Apr. 1, 2014.

### FIELD OF THE INVENTION

The present invention relates generally to a medical device for monitoring the levels of biomarkers in bodily fluid. More specifically, the present invention is a biomarker detection device that is utilized to acquire and analyze an acquired sample of bodily fluid for the presence and levels of various peptide and non-peptide biomarkers. The present invention is primarily described in relation the heart failure biomarker Brain or B-type Natriuretic Peptide (BNP), but may be utilized for monitoring additional peptide and non-peptide biomarkers as well.

### BACKGROUND OF THE INVENTION

In the medical field, a biomarker is a measurable parameter that is indicative of the presence of a disease, condition, or other medical state. Biomarkers are typically present and may be measured in biological samples such as whole blood, interstitial fluid, and/or plasma. There are several classes of biomarkers including biomarkers that are indicative of a disease status or risk, biomarkers that measure the effect of a drug or other substance introduced into a biological organism, and biomarkers that measure the direct effect of a drug on a target molecule or receptor. An example biomarker is the heart failure biomarker Brain or B-type Natriuretic Peptide (BNP). This biomarker is a polypeptide that is secreted by the heart ventricles in response to excessive stretching, straining, or impairment of the heart muscles. Elevated levels of BNP in the blood are often indicative of the impending onset of heart failure. The conventional method of detecting biomarkers for the purpose of monitoring a medical condition typically requires large volume samples of blood, interstitial fluid, plasma, or other bodily fluids to be drawn from a patient. Analysis is performed on these samples in order to detect the presence of biomarkers as well as the levels of biomarkers. The measured levels of biomarkers are then compared against standardized normal levels in order to identify any anomalies. The sample-acquisition process is usually painful for patients as large quantities of bodily fluids must be drawn using large bore needles. Simple testing is sometimes conducted on a smaller scale using smaller equipment at self-contained facilities such as emergency rooms. However, this is often simply not possible for more elaborate or more specialized testing. Such specialized testing generally requires that samples be transported to centralized facilities, at times over long distances and requiring a waiting time of several days.

The present invention is a biomarker detection device that is capable of acquiring and analyzing a microfluidic sample for the presence and levels of peptide and non-peptide biomarkers. The biomarker detection device is utilized to acquire and analyze samples on the micro to nano scale volumes, eliminating the need for large volume patient samples. The present invention is primarily designed to detect the presence of and measure the heart failure biomarker BNP although the present invention may additionally be utilized to detect and measure a variety of additional peptide and non-peptide biomarkers. The biomarker detection device may be worn by the user and is entirely self-contained. An acquired microfluidic sample is mixed with a reagent in order to allow antibodies within the reagent to specifically bind to any peptide and non-peptide biomarkers in the sample of bodily fluid. The biomarker detection device is then able to determine the levels of the biomarkers in the bodily fluid and compare the sample values against standardized normal levels for the biomarkers within the sample in order to detect or monitor the status of various conditions of interest such as heart failure. The biomarker detection device then provides the results of the analysis to the user and results may be wirelessly transmitted to an external device or source.

### BRIEF DESCRIPTION OF THE DRAWINGS

FIG. 1 is a perspective view of the present invention.

FIG. 2A is a left side view of the present invention.

FIG. 2B is an example diagrammatic view of the microneedle array and the H-filter of the present invention.

FIG. 3 is a diagrammatic overview of electronic connections of the present invention.

FIG. 4 is an example diagrammatic view of the microneedle array and the H-filter with the retractable cover in place over the microneedle array.

FIG. 5 is an example diagrammatic view of the microneedle array and the H-filter with the retractable cover removed from the microneedle array.

FIG. 6 is an example diagrammatic view of the microneedle and the H-filter detailing the fluid dynamics of the present invention.

FIG. 7 is an example diagrammatic view of the H-filter detailing particle movement in the H-filter.

### DETAIL DESCRIPTIONS OF THE INVENTION

All illustrations of the drawings are for the purpose of describing selected versions of the present invention and are not intended to limit the scope of the present invention.

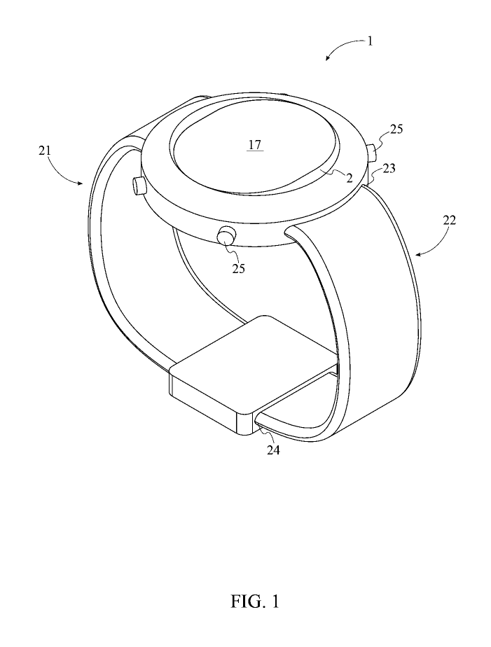

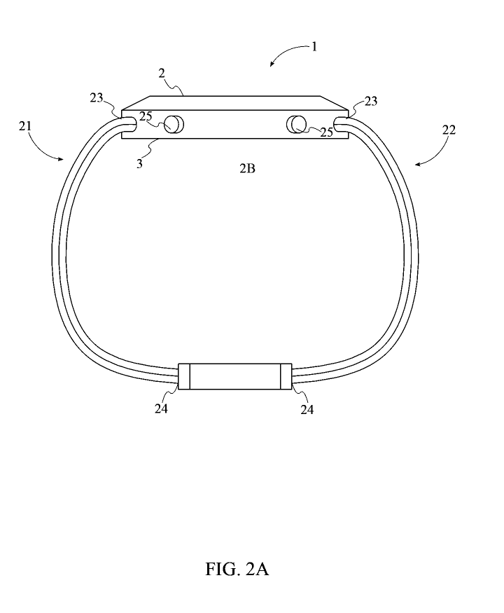

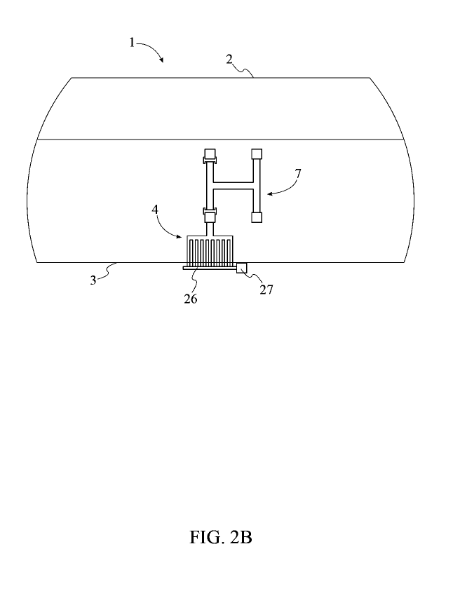

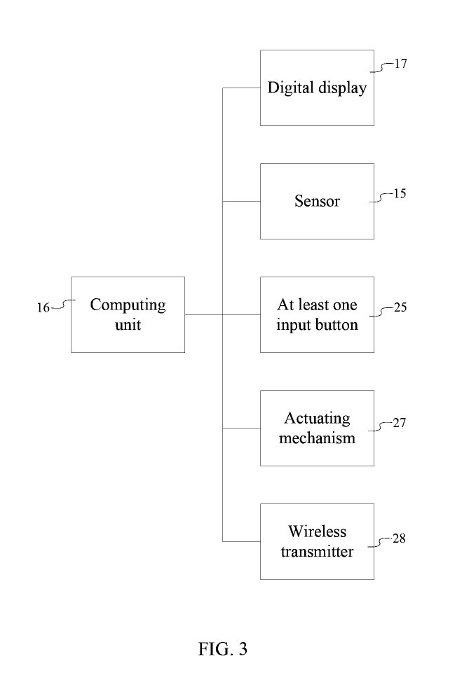

The present invention is a biomarker detection device for acquiring and analyzing a sample of bodily fluid in order to monitor various peptide and non-peptide biomarkers 100. The present invention comprises a wearable case 1, a microneedle array 4, an H-filter 7, a sensor 15, a computing unit 16, and a digital display 17. The wearable case 1 is shown in FIG. 1 and FIG. 2A. The wearable case 1 is worn by the user and placed into contact with the user's skin. The microneedle array 4 may be inserted into the user's skin in order to acquire a sample of bodily fluid on the micro or nano scale from the patient's body without the need for potentially painful large bore needles. The H-filter 7 is utilized to separate a particular constituent or subset of constituents of the heterogeneous sample of bodily fluid from the remaining constituents. The sensor 15 is able to identify the presence of certain biomarkers 100 in the sample in order to determine if the biomarkers 100 are indicative of an imminent or potential future medical issue. The sensor 15 is electronically connected to the computing unit 16 in order to allow the computing unit 16 to perform analysis on the content properties of the sample of bodily fluid. Electronic connections of the present invention are shown in FIG. 3. Any results of the analysis and additional information provided by the present invention are output to the digital display 17 for viewing by the user. In various example embodiments of the present invention, the digital display 17 may feature touchscreen technology for user touch input.

Again referring to FIG. 1 and FIG. 2A, the wearable case 1 comprises a first surface 2 and a second surface 3 with the first surface 2 and the second surface 3 being positioned opposite of each other. In the preferred embodiment of the present invention, the present invention is wearable in the same manner as a conventional wristwatch. The digital display 17 is mounted into the first surface 2 and is viewable by the user while the present invention is worn on the user's wrist. The second surface 3 is oriented toward the user's skin.

The H-filter 7 comprises a first input tube 8 and a first sorting tube 11. The H-filter 7 and the computing unit 16 are enclosed within the wearable case 1 in order to protect the H-filter 7 and the computing unit 16 from potential damage during use. The first input tube 8 is in fluid communication with the microneedle array 4. A sample of bodily fluid that is drawn from the user by the microneedle array 4 is directed into the H-filter 7 through the first input tube 8. The first sorting tube 11 is in fluid communication with the first input tube 8. The sensor 15 is mounted adjacent to the first sorting tube 11, enabling the sensor 15 to come into contact with the acquired sample of bodily fluid through the first input tube 8 and the first sorting tube 11. The sensor 15 is electronically connected to the computing unit 16 with sample information acquired through the sensor 15 analyzed by the computing unit 16. The computing unit 16 is electronically connected to the digital display 17 and the user is able to view analyzed sample information on the digital display 17.

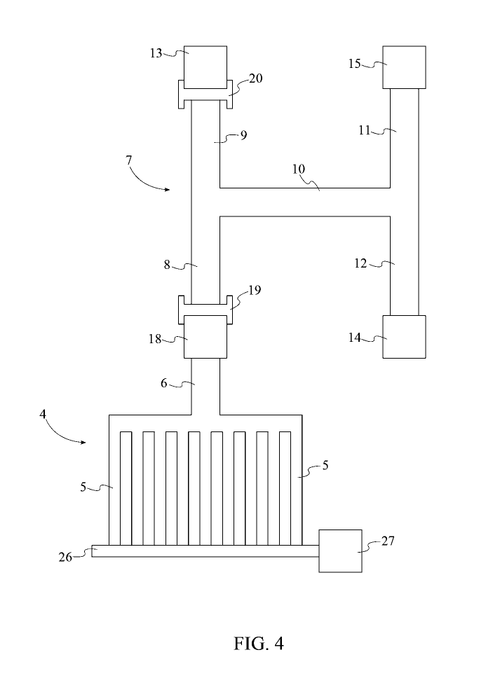

As shown in FIG. 2B, the microneedle array 4 is adjacently mounted to the second surface 3 in order to allow the microneedle array 4 to pierce the user's skin prior to and during acquisition of a bodily fluid sample. The microneedle array 4 is hollow in order to allow a sample of bodily fluid to flow through the plurality of needles. Additionally, the microneedle array 4 may feature an anticoagulant such as heparin in order to minimize the likelihood of the microneedle array 4 becoming obstructed. As shown in FIG. 4, the microneedle array 4 comprises a plurality of microneedles 5 and a microneedle hub 6. Each of the plurality of microneedles 5 is in fluid communication with the microneedle hub 6. The plurality of microneedles 5 is designed to penetrate into the dermal layer of skin during sample acquisition. Each of the plurality of microneedles 5 is in fluid communication with the microneedle hub 6. The plurality of microneedles 5 converges into the microneedle hub 6 with the microneedle hub 6 positioned in between the second surface 3 and the plurality of microneedles 5. A bodily fluid sample that is drawn from the user enters the microneedle array 4 through the plurality of microneedles 5 and is directed into the microneedle hub 6. Because the first input tube 8 is in fluid communication with the microneedle array 4, the bodily fluid sample is then directed from the microneedle array 4 into the H-filter 7.

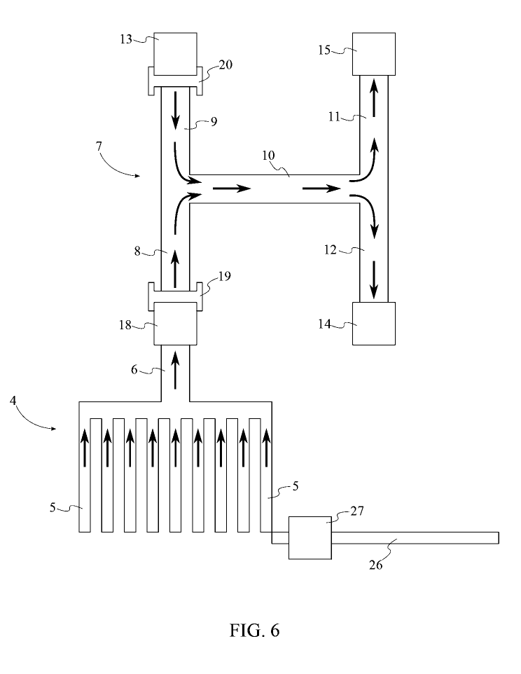

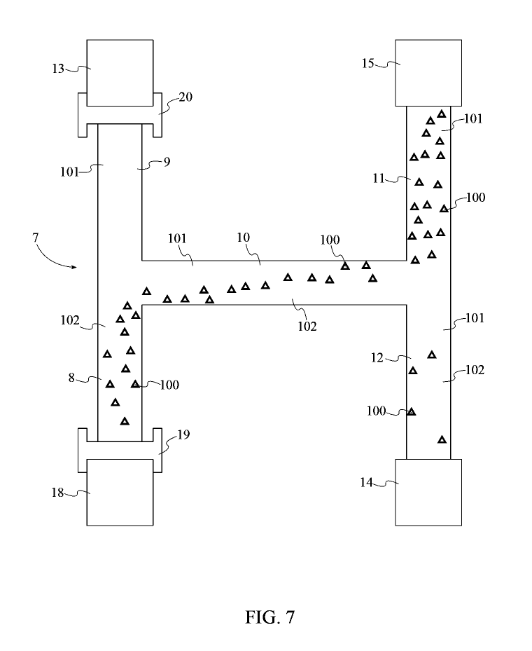

The H-filter 7 further comprises a second input tube 9, a diffusion tube 10, a second sorting tube 12, a reagent chamber 13, a sample preparation chamber 18, and a waste receptacle 14. The diffusion tube 10 is in fluid communication with the microneedle array 4 through the first input tube 8. As such, a bodily fluid sample is able to enter the diffusion tube 10 by entering the H-filter 7 from the microneedle array 4 through the first input tube 8. The diffusion tube 10 is in fluid communication with the reagent chamber 13 through the second input tube 9. The reagent chamber 13 contains antibodies 101 that are capable of identifying and binding to various biomarkers 100 within an acquired sample of bodily fluid. The sample of bodily fluid and the reagent enter the diffusion chamber where the bonded antibodies 101 and biomarkers 100 are able to separate from interfering particles 102 in the sample of bodily fluid. The sensor 15 is in fluid communication with the diffusion tube 10 through the first sorting tube 11. Once the sample of bodily fluid and the reagent have entered the diffusion tube 10, the bonded antibodies 101 and biomarkers 100 are able to separate from the remaining mixture and are diffused into the first sorting tube 11 as shown in FIG. 6 and FIG. 7. The sensor 15 is then able to detect the presence of biomarkers 100 through the presence of the antibodies 101 that have bonded to the biomarkers 100. The waste receptacle 14 is in fluid communication with the diffusion tube 10 through the second sorting tube 12. The waste receptacle 14 serves to contain waste fluid and interfering particles 102 that are not considered relevant to the sensor 15 detecting the presence of biomarkers 100 in the user's bodily fluid sample. Waste fluid and interfering particles 102 exiting the diffusion tube 10 are able to enter the waste receptacle 14 through the second sorting tube 12. The waste receptacle 14 may be removable in various embodiments of the present invention.

In an embodiment of the present invention, the second input tube 9 is collinear with the first input tube 8 while the second sorting tube 12 is collinear with the first sorting tube 11. The diffusion tube 10 is positioned perpendicular to and in between the first sorting tube 11 and the second sorting tube 12. Additionally, the first input tube 8 and the second input tube 9 are positioned opposite to the first sorting tube 11 and the second sorting tube 12 across the diffusion tube 10. This arrangement of the first input tube 8, the second input tube 9, the diffusion tube 10, the first sorting tube 11, and the second sorting tube 12 forms the “H” design of the H-filter 7. Additionally, the arrangement separates the first input tube 8 and the second input tube 9 from the first sorting tube 11 and the second sorting tube 12 via the diffusion tube 10. This allows the sample of bodily fluid and the reagent to come into contact with each other within the diffusion tube 10 before the bonded antibodies 101 and biomarkers 100 are separated and directed towards the sensor 15 through the first sorting tube 11.

The present invention further comprises a sample preparation chamber 18. The microneedle array 4 is in fluid communication with the first input tube 8 through the sample preparation chamber 18. The sample preparation chamber 18 modifies the acquired sample of bodily fluid in order to improve bonding interactions between the antibodies 101 and the biomarkers 100. The interior of the sample preparation chamber 18 may feature an anticoagulant such as heparin in order to minimize the likelihood of obstruction of the H-filter 7. This process is conducted prior to the sample of bodily fluid coming into contact with the reagent in order to ensure that the sample of bodily fluid and the reagent are able to properly mix within the diffusion chamber. As such, the sample preparation chamber 18 is fluidly integrated in between the microneedle array 4 and the first input tube 8. This further ensures that the bonded antibodies 101 and biomarkers 100 are able to separate from the remaining interfering particles 102 in the mixture.

The present invention further comprises a first pumping mechanism 19 and a second pumping mechanism 20. The first pumping mechanism 19 is positioned in between the microneedle array 4 and the first input tube 8 with the first input tube 8 being in fluid communication with the microneedle array 4 through the first pumping mechanism 19. The first pumping mechanism 19 is activated upon the plurality of microneedles 5 being inserted into the user's skin and serves to draw a micro sample of bodily fluid into the microneedle array 4. The second pumping mechanism 20 is positioned in between the reagent chamber 13 and the second input tube 9 with the second input tube 9 in fluid communication with the reagent chamber 13 through the second pumping mechanism 20. The second pumping mechanism 20 is utilized to release reagent into the second input tube 9.

The present invention further comprises a first strap 21 and a second strap 22. The first strap 21 and the second strap 22 are utilized to join the present invention to the user's body, preferably to the user's wrist. The first strap 21 and the second strap 22 are composed of a flexible material that may be wrapped around the user's wrist. The first strap 21 and the second and each comprise a distal end 23 and a proximal end 24. The proximal end 24 of the first strap 21 is hingedly and adjacently connected to the wearable case 1, opposite to the proximal end 24 of the first strap 21. The proximal end 24 of the second strap 22 is hingedly and adjacently connected to the wearable case 1, opposite to the proximal end 24 of the first strap 21. This allows the first strap 21 and the second strap 22 to be wrapped around the user's wrist. The distal end 23 of the first strap 21 and the distal end 23 of the second strap 22 are detachably coupled to each other. As such, the distal end 23 of the first strap 21 and the distal end 23 of the second strap 22 may be coupled to each other in order to secure the present invention to the user's wrist while allowing the user to remove the present invention as needed when the present invention is not in used.

The present invention further comprises at least one physical input 25. The at least one physical input 25 allows the user to access the various functions of the present invention. User commands are provided to the computing unit 16 through the at least one physical input 25. The at least one physical input 25 is laterally mounted into the wearable case 1 in order to provide convenient access to the at least one physical input 25 for the user. Additionally, the at least one physical input 25 is electronically connected into the computing unit 16 and the computing unit 16 is able to interpret user commands that are provided via the at least one physical input 25.

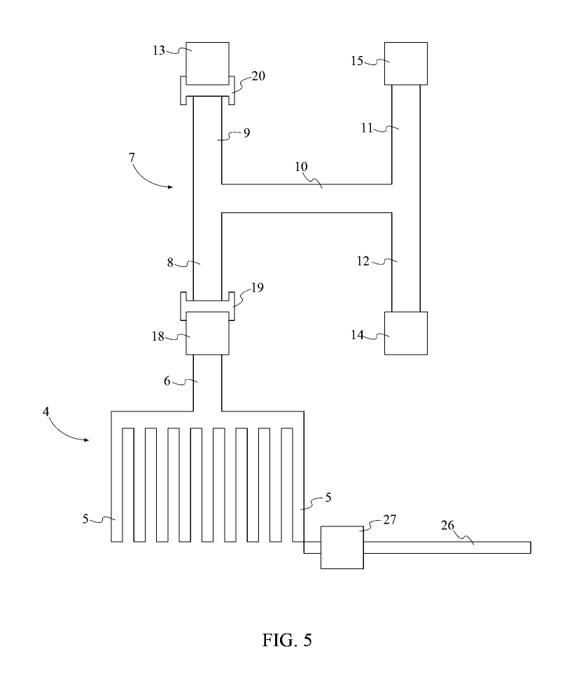

The plurality of microneedles 5 is designed to pierce the user's skin during sample acquisition. The present invention may be utilized to acquire and test a sample on a timed (interval) basis, on a continuous monitoring basis, or only when the user wishes. As such, it is desirable that the plurality of microneedles 5 is not continuously embedded in the user's skin when the present invention is not in use. The present invention further comprises a retractable cover 26 and an actuating mechanism 27. The retractable cover 26 is selectively positioned adjacent to the plurality of microneedles 5, opposite to the second surface 3. As shown in FIG. 5, the retractable cover 26 is slidably mounted to the wearable case 1 and is utilized to cover or uncover the plurality of needles as needed by the user. The actuating mechanism 27 is operatively coupled to the retractable cover 26 and electronically connected to the computing unit 16. As such, the user is able to provide a command through the at least one physical input 25 in order to allow the computing unit 16 to engage or disengage the retractable cover 26 via the actuating mechanism 27.

The present invention further comprises a communications module 28. The communications module 28 is enclosed within the wearable case 1 in order to prevent the communications module 28 from becoming damaged during use of the present invention. In an embodiment of the present invention, the communications module 28 is electronically connected to the computing unit 16 and is utilized to wirelessly transmit results obtained from the analysis of an acquired sample of patient bodily fluid to an external electronic device or party, for example, to a medical professional.

The object of the present invention is to detect the presence of biomarkers 100 in an acquired sample of bodily fluid. Biomarkers 100 are commonly indicative of various medical conditions. For example, the presence of the biomarker Brain or B-type Natriuretic Peptide (BNP) is often indicative of imminent heart failure. The sensor 15 and the computing unit 16 of the present invention are capable of detecting and analyzing the presence of biomarkers 100 in an acquired sample. If biomarker levels in the sample are considered to be unsafe or outside of a “normal” range, the computing unit 16 is able to notify the user through an alert system. The present invention eliminates the need for large volume bodily fluid samples to be drawn from the user as well.

Although the invention has been explained in relation to its preferred embodiment, it is to be understood that many other possible modifications and variations can be made without departing from the spirit and scope of the invention as hereinafter claimed.

What is claimed is:

1. A biomarker detection device comprises:
a wearable case;
a microneedle array;
an H-filter;
a sensor;
a computing unit;
a digital display;
the wearable case comprises a first surface and a second surface;
the H-filter comprises a first input tube and a first sorting tube;
the first surface and the second surface being positioned opposite to each other;
the digital display being mounted into the first surface;
the microneedle array being adjacently mounted to the second surface;
the H-filter and the computing unit being enclosed within the wearable case;
the first input tube being in fluid communication with the microneedle array;
the first sorting tube being in fluid communication with the first input tube;
the sensor being mounted adjacent to the first sorting tube;
the sensor being electronically connected to the computing unit;
the computing unit being electronically connected to the digital display;

2. The biomarker detection device as claimed in claim 1 further comprises:
the microneedle array comprises a plurality of microneedles and a microneedle hub;
each of the plurality of microneedles being in fluid communication with the microneedle hub;
the microneedle hub being positioned in between the second surface and the plurality of microneedles;

3. The biomarker detection device as claimed in claim 1 further comprises:
the H-filter further comprises a second input tube, a diffusion tube, a second sorting tube, a reagent chamber, a sample preparation chamber, and a waste receptacle;
the diffusion tube being in fluid communication with the microneedle array through the first input tube;
the diffusion tube being in fluid communication with the reagent chamber through the second input tube;
the sensor being in fluid communication with the diffusion tube through the first sorting tube;
the waste receptacle being in fluid communication with the diffusion tube through the second sorting tube;

4. The biomarker detection device as claimed in claim 2 further comprises:
the second input tube being collinear with the first input tube;
the second sorting tube being collinear with the first sorting tube;
the diffusion tube being positioned perpendicular to and in between the first input tube and the second input tube;
the diffusion tube being positioned perpendicular to and in between the first sorting tube and the second sorting tube;
the first input tube and the second input tube being positioned opposite to the first sorting tube and the second sorting tube across the diffusion tube;

5. The biomarker detection device as claimed in claim 1 further comprises:
a sample preparation chamber;
the microneedle array being in fluid communication with the first input tube through the sample preparation chamber;
the sample preparation chamber being fluidly integrated in between the microneedle array and the sample preparation chamber;

6. The biomarker detection device as claimed in claim 1 further comprises:
a first pumping mechanism;
a second pumping mechanism;
the H-filter further comprises a first input tube, a second input tube, and a reagent chamber;
the first pumping mechanism being positioned in between the microneedle array and the first input tube;
the first input tube being in fluid communication with the microneedle array through the first pumping mechanism;
the second pumping mechanism being positioned in between the reagent chamber and the second input tube;
the second input tube being in fluid communication with the reagent chamber through the second pumping mechanism;

7. The biomarker detection device as claimed in claim 1 further comprises:
a first strap;
a second strap;
the first strap and the second strap each comprise a distal end and a proximal end;
the proximal end of the first strap being hingedly and adjacently connected to the wearable case;
the proximal end of the second strap being hingedly and adjacently connected to the wearable case, opposite to the proximal end of the first strap;
the distal end of the first strap and the distal end of the second strap being detachably coupled to each other;

8. The biomarker detection device as claimed in claim 1 further comprises:
at least one physical input;
the at least one physical input being laterally mounted into the wearable case;
the at least one physical input being electronically connected to the computing unit;

9. The biomarker detection device as claimed in claim 1 further comprises:
a retractable cover;
an actuating mechanism;
the retractable cover being selectively positioned adjacent to a plurality of microneedles of the microneedle array, opposite to the second surface;
the retractable cover being slidably mounted to the wearable case;
the actuating mechanism being operatively coupled to the retractable cover;
the actuating mechanism being electronically connected to the computing unit;

10. The biomarker detection device as claimed in claim 1 further comprises:
a communications module;
the communications module being enclosed within the wearable case;
the communications module being electronically connected to the computing unit;

11. A biomarker detection device comprises:
a wearable case;
a microneedle array;
an H-filter;
a sensor;
a computing unit;
a digital display;
a sample preparation chamber;
the wearable case comprises a first surface and a second surface;
the H-filter comprises a first input tube and a first sorting tube;
the first surface and the second surface being positioned opposite to each other;
the digital display being mounted into the first surface;
the microneedle array being adjacently mounted to the second surface;
the H-filter and the computing unit being enclosed within the wearable case;
the first input tube being in fluid communication with the microneedle array;
the first sorting tube being in fluid communication with the first input tube;
the sensor being mounted adjacent to the first sorting tube;
the sensor being electronically connected to the computing unit;
the computing unit being electronically connected to the digital display;
the microneedle array being in fluid communication with the first input tube through the sample preparation chamber;
the sample preparation chamber being fluidly integrated in between the microneedle array and the sample preparation chamber;

12. The biomarker detection device as claimed in claim 11 further comprises:
the microneedle array comprises a plurality of microneedles and a microneedle hub;
each of the plurality of microneedles being in fluid communication with the microneedle hub;
the microneedle hub being positioned in between the second surface and the plurality of microneedles;

13. The biomarker detection device as claimed in claim 11 further comprises:
the H-filter further comprises a second input tube, a diffusion tube, a second sorting tube, a reagent chamber, a sample preparation chamber, and a waste receptacle;
the diffusion tube being in fluid communication with the microneedle array through the first input tube;
the diffusion tube being in fluid communication with the reagent chamber through the second input tube;
the sensor being in fluid communication with the diffusion tube through the first sorting tube;
the waste receptacle being in fluid communication with the diffusion tube through the second sorting tube;
the second input tube being collinear with the first input tube;
the second sorting tube being collinear with the first sorting tube;
the diffusion tube being positioned perpendicular to and in between the first input tube and the second input tube;
the diffusion tube being positioned perpendicular to and in between the first sorting tube and the second sorting tube;
the first input tube and the second input tube being positioned opposite to the first sorting tube and the second sorting tube across the diffusion tube;

14. The biomarker detection device as claimed in claim 11 further comprises:
a first pumping mechanism;
a second pumping mechanism;
the H-filter further comprises a first input tube, a second input tube, and a reagent chamber;
the first pumping mechanism being positioned in between the microneedle array and the first input tube;
the first input tube being in fluid communication with the microneedle array through the first pumping mechanism;
the second pumping mechanism being positioned in between the reagent chamber and the second input tube;
the second input tube being in fluid communication with the reagent chamber through the second pumping mechanism;

15. The biomarker detection device as claimed in claim 11 further comprises:
a first strap;
a second strap;
the first strap and the second strap each comprise a distal end and a proximal end;
the proximal end of the first strap being hingedly and adjacently connected to the wearable case;
the proximal end of the second strap being hingedly and adjacently connected to the wearable case, opposite to the proximal end of the first strap;
the distal end of the first strap and the distal end of the second strap being detachably coupled to each other;

16. The biomarker detection device as claimed in claim 11 further comprises:
at least one physical input;
the at least one physical input being laterally mounted into the wearable case;
the at least one physical input being electronically connected to the computing unit;

17. The biomarker detection device as claimed in claim 11 further comprises:
a retractable cover;
an actuating mechanism;
the retractable cover being selectively positioned adjacent to a plurality of microneedles of the microneedle array, opposite to the second surface;
the retractable cover being slidably mounted to the wearable case;
the actuating mechanism being operatively coupled to the retractable cover;
the actuating mechanism being electronically connected to the computing unit;

18. The biomarker detection device as claimed in claim 11 further comprises:
a communications module;
the communications module being enclosed within the wearable case;
the communications module being electronically connected to the computing unit;
